(19) United States
(12) Patent Application Publication
(10) Pub. No.: US 2005/0209624 A1
(43) Pub. Date: Sep. 22, 2005

(54) Scissors for piercing and cutting anatomical vessels

(76) Inventor: Venkataramana Vijay, Tarrytown, NY (US)

Correspondence Address: David S. Jacobson;Gordon & Jacobson, P.C., 65 Woods End Road, Stamford, CT, 06905, US

(21) Appl. No.: 10/805,693

(22) Filed: Mar. 22, 2004

(51) Int. Cl. A61B 17/32

(52) U.S. Cl. 606/174

(57) ABSTRACT

A surgical scissors includes two cutting blades operable via a handle. One of the blades is provided with a distally projecting needle-like tip which can easily pierce tissue. In use, the tip on the blade is gently pressed against a vessel, preferably at a shallow angle relative to the vessel, to define an entry hole. This shallow angle approach with the needle-like piercing tip reduces the opportunity for inadvertent puncture. The lower blade is then pushed further through the entry hole in alignment with the piercing tip such that the cutting blade portion of the lower blade also enters the vessel. The handle is then operated to cause the blades cut the vessel tissue therebetween to create the incision. The instrument of the invention facilitates making lengthwise incisions in vessels by eliminating offline cutting, and substantially reducing the likelihood of cutting the posterior vessel wall.

### BACKGROUND OF THE INVENTION

1. Field of the Invention

This invention relates broadly to surgical instruments. More particularly, this invention relates to a scissors device for piercing and cutting vessels such as blood vessels.

2. State of the Art

During various surgical procedures, a portion of a small vessel must be incised lengthwise. Such an incision requires two steps. A standard scalpel is first used to make a small hole in the anterior vessel wall, and then a vascular scissors instrument 10 is used to make a lengthwise cut starting at the small hole. Referring to prior art FIG. 1, the scissors instrument 10 includes upper and lower blades 12, 14 and a handle 14 operable to move the blades relative to each other. The lower blade 14 of the scissors is inserted through the small hole, and the handle is operated to cause the blades to cut lengthwise along the vessel to create an incision of the required length. This two-step process is particularly delicate for very small vessels, e.g., on the order of 2 mm in diameter and smaller.

For example, incisions on small vessels are required in various vascular, cardiac, ophthalmic, urethral, and fallopian procedures. In each case, if during the initial cut the scalpel is inadvertently pressed too far into the vessel during creation of the incision entry, serious damage can result to the posterior surface of the vessel as well as the underlying tissue. In many cases, for example during cardiac procedures, this damage can be very serious, and even life threatening.

Microvascular scissors do have sharp points, but are not designed small enough to pierce the vessel wall without ripping it. In addition, the length and taper of the two blades of the microvascular scissors are identical. Therefore, as the lower blade is forced to pierce the vessel, the tip of the upper blade invariably pierces or rips the adjoining anterior portion of the vessel. Further, the diameter of the lower blade in a microvascular scissors widens dramatically thereby preventing travel into the vessel lumen, especially if the vessel is of small diameter (e.g., 2 mm to 4 mm), which is frequently the case with coronary vessels.

### SUMMARY OF THE INVENTION

It is therefore an object of the invention to provide an instrument which facilitates making lengthwise incisions on small vessels.

It is another object of the invention to provide an instrument which dramatically reduces the risk of inadvertent damage to small vessels and the patient.

In accord with these objects, which will be discussed in detail below, a surgical scissors instrument includes upper and lower tissue cutting blades and a handle manually operable to move one blade relative to the other between open and closed positions in a scissoring action. The upper blade preferably has a concave bow or is slightly angled relative to the lower blade which causes the lower surface of the upper blade and the upper surface of the lower blade to contact each other as the handle is operated and define a cutting interface.

In accord with the invention, one of the blades and most preferably the lower blade is provided with a distally projecting tip sufficiently sharp to relatively easily pierce tissue. The tip is preferably a round body needle or tear drop-shape cutting edge needle.

The blades and/or the tip may be permanently attached to the scissors, may be removable and re-sharpened, may be disposable and replaceable, and may be made from metal and/or non-metal components. In addition, the blades may be retractable.

When in a closed position, the upper and lower blades are preferably angulated relative to the axis of the handle, e.g., either at 30°, 45°, 60° or 90°. The handle, whether metal or non-metal, permanent or disposable, may be of any suitable type, such as Castroviejos type or a common two ring handle. These cutting blades may also be coupled to a long handle to allow manual or robotic thoracoscopic or endoscopic use or on a catheter for percutaneous application.

In use, the piercing tip on the lower blade is gently pressed against a vessel, preferably at a shallow angle relative to the vessel, to define an entry hole. This shallow angle approach with the needle-like piercing tip reduces the opportunity for inadvertent puncture. The lower blade is then pushed further through the entry hole in alignment with the piercing tip such that the cutting blade portion of the lower blade also enters the vessel. The handle is then operated to cause the upper blade to rotate relative to the lower blade to cut the vessel tissue therebetween to create the incision. If necessary, the blades can be opened and moved to cut additional vessel tissue.

The instrument of the invention facilitates making lengthwise incisions in vessels by eliminating offline cutting, and substantially reducing the likelihood of cutting the posterior vessel wall. Furthermore, a procedure that previously required two instruments and at least two steps can now be performed more safely with a single instrument and in a single step. In addition, the instrument is particularly useful in both manual and robotic procedures where the need to change instruments during a procedure in reduced.

Additional objects and advantages of the invention will become apparent to those skilled in the art upon reference to the detailed description taken in conjunction with the provided figures.

### BRIEF DESCRIPTION OF THE DRAWINGS

Prior art FIG. 1 shows a scissors with a Castroviejos handle of the type conventionally used in vascular surgery;

FIG. 2 is a side elevation of a first embodiment of a scissors of the invention;

FIG. 3 is a side elevation of a second embodiment of a scissors of the invention;

FIG. 4 is a broken side elevation of a third embodiment of a scissors of the invention;

FIG. 5 is a side elevation of a fourth embodiment of a scissors of the invention; and

FIG. 6 is a side elevation of a fifth embodiment of a scissors of the invention.

### DETAILED DESCRIPTION OF THE PREFERRED EMBODIMENTS

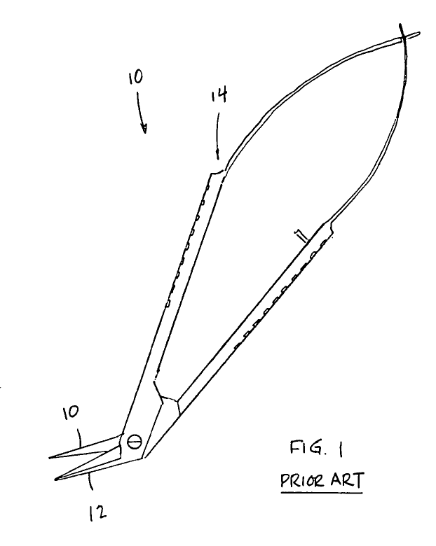

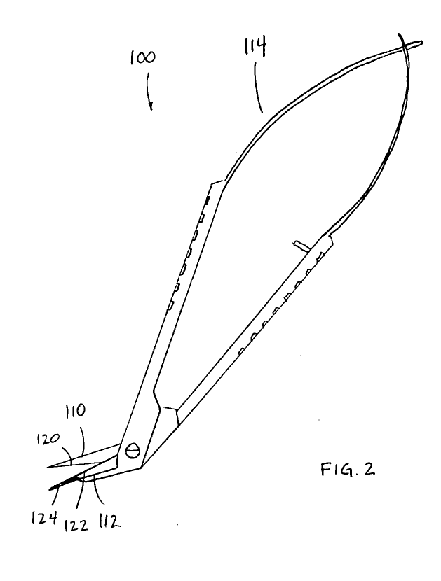

Turning now to FIG. 2, a surgical scissors instrument 100 includes upper and lower tapering tissue cutting blades 110, 112 and a handle 114 manually operable to move one blade relative to the other between open and closed positions in a scissoring action. The handle 114 may be a Castroviejos-type spring handle (as shown) or a well-known ring handle (with rings or similar structure for fingers to manipulate the handle).

The upper blade 110 is preferably relative planar along its cutting surface. The lower blade 112 preferably has a concave bow or is slightly angled relative to the upper blade 110 which causes the upper surface 122 of the lower blade 112 and the lower surface 120 of the upper blade 110 to contact each other as the handle 114 is operated so as to define a cutting interface.

In accord with the invention, one of the blades and most preferably the lower blade 112 is provided with a distally projecting tip 124 sufficiently sharp and long to relatively easily pierce tissue. The tip 124 preferably has a constant or suitable variable diameter over its length. The tip 124 is preferably a round body needle or needle having a triangular or tear-drop sectioned cutting edge shape. The needle gauge is variable along its shaft, depending upon the size of the vessel being opened. However, the ground point of the needle is preferably a 25 or 26 gauge needle facilitating penetration of vessels of the small size.

According to a first embodiment of the invention, the lower blade 112 tapers or steps down to at least partially define the tip 124 such that the upper blade 110 and the tip when in a closed position terminate at substantially a common location. The taper preferably, though optionally not exclusively, occurs from a lower portion of blade 112 so that upper surface 122 of the lower blade 112 remains straight and continuous along the entirety of the blade; i.e., the cutting edge of the length of the lower blade 112, including tip 124, is identical to that of the upper blade 110.

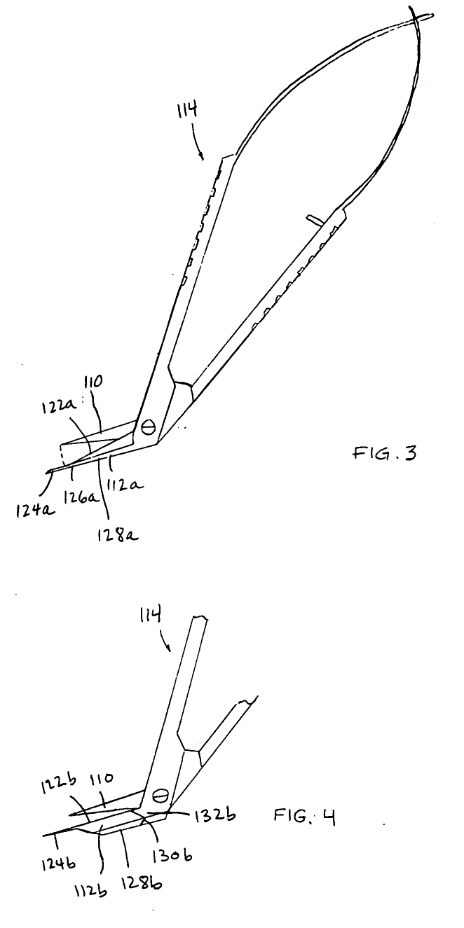

Turning now to FIG. 3, according to a second embodiment of the invention, the upper and lower blades 110, 112a have shapes substantially similar to that of prior art instruments. In accord with the invention, the tip 124a extends from the distal end 126a of the lower blade 112a, such that when the upper and lower blades 110, 112a are in a closed position, the tip extends further therefrom. In the embodiment of FIG. 3, the tip 124a is aligned with the lower surface 128a of the lower blade 112a. In the alternative, the tip 124a may extend in alignment with the upper surface 122a of the lower blade, or in some other direction.

Turning now to FIG. 4, according to a third embodiment, which is substantially a combination of the first and second embodiments, the cutting edge of the lower blade 112b is continuous with the tip 124b, and the tip extends from the distal end 126b of the lower blade 112b, such that when the upper and lower blades 110, 112b are in a closed position, the tip 124b extends further therefrom. The tip 124b may be defined by a tapering of the lower surface 128b of the lower blade. In addition, 130b identifies a location for detachment of the lower blade 112b which, e.g., may be coupled to the remaining blade arm 132b via a friction fit or snap fit, permitting the use of a disposable blade and tip.

When in a closed position, the upper and lower blades are preferably angulated relative to the axis of the handle, e.g., either at 30°, 45° (as shown), 60° or 90°.

The blades 110, 112 (referring hereinafter also to 112a and 112b) and/or the tip 124 (referring hereinafter also to 124a and 124b) may be permanently attached to the scissors 100, may be removable and re-sharpened, may be disposable and replaceable, and may be made from metal and/or non-metal components. The blades may also be retractable.

In use, the piercing tip 124 on the lower blade 112 is gently pressed against an anterior surface of a vessel, preferably at a shallow angle relative to the vessel, to define an entry hole. This shallow angle approach with the needle-like piercing tip 124 reduces the opportunity for inadvertent puncture. With the embodiment of FIG. 2, the handle 114 is then operated to cause the upper blade 110 to rotate relative to the lower blade 112 to cut the vessel tissue therebetween to create the incision. With the embodiments of FIGS. 3 and 4, the lower blade 112a, 112b is preferably advanced further through the entry hole in alignment with the piercing tip 124a, 124b such that the upper surface 122a, 122b of the cutting blade 112a, 112b also enters the vessel. Then the handle 114 is operated to cut the tissue. If necessary, the blades 110, 112 can be opened and moved to cut additional vessel tissue.

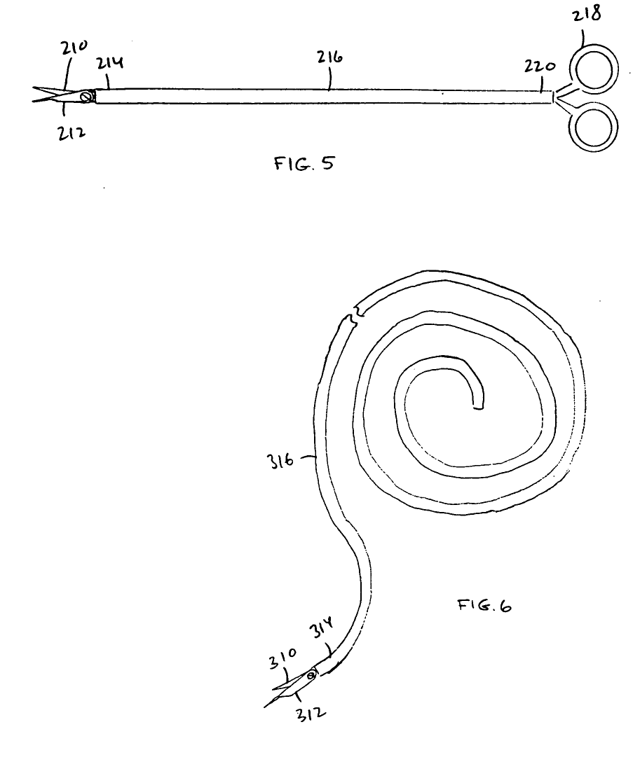

Turning to FIG. 5, the cutting blades 210, 212 of the invention may be coupled to the distal end 214 of a long shaft 216 (flexible or rigid) and operated by manual operation, e.g., via a handle 218 (other handles can be used), or robotic operation, at a proximal end 220 of the shaft. Apart from the blades 210, 212 of the invention, such thoracoscopic or endoscopic scissors are well-known. For example, U.S. Pat. No. 5,392,789, which is incorporated by reference herein in its entirety, teaches an endoscopic scissors. It is appreciated that in such an embodiment, the blades 210, 212 are preferably in-line with the shaft 216.

Referring now to FIG. 6, the cutting blades 310, 312 of the invention may also be coupled to the distal end 314 of a catheter 316 or another similar flexible tubular construct for percutaneous application. For example, U.S. Pat. Nos. 5,817,013 and 6,352,503, which are incorporated by reference herein in their entireties, teach scissors blades coupled to the distal end of a flexible tubular member.

The instrument of the invention facilitates making lengthwise incisions in vessels by eliminating offline cutting, and substantially reducing the likelihood of cutting the posterior vessel wall. Furthermore, a procedure that previously required two instruments can now be performed more safely with a single instrument.

There have been described and illustrated herein embodiments of a surgical scissors and a method of using the same. While particular embodiments of the invention have been described, it is not intended that the invention be limited thereto, as it is intended that the invention be as broad in scope as the art will allow and that the specification be read likewise. It will therefore be appreciated by those skilled in the art that yet other modifications could be made to the provided invention without deviating from its spirit and scope as claimed.

1. A scissors instrument, comprising:
a) a first blade;
b) a second blade defining a cutting interface with said first blade;
c) a tissue piercing tip of substantially smaller diameter than said first and second blades projecting from one of said first and second blades; and
d) a handle operable to move said first and said blades relative to each other.

2. A scissors instrument according to claim 1, wherein:
said tip is grounded so as to be not larger than approximately a 25 gauge needle.

3. A scissors instrument according to claim 1, wherein:
said first blade is an upper blade, said second blade is a lower blade, and said tip is coupled to said lower blade.

4. A scissors instrument according to claim 3, wherein:
said tip extends beyond said first blade.

5. A scissor instrument according to claim 3, wherein:
said first blade, and second blade and tip coupled thereto are of substantially equal length.

6. A scissors instrument according to claim 1, wherein:
said second blade includes an upper cutting surface, and said tip is substantially parallel to said upper cutting surface.

7. A scissors instrument according to claim 6, wherein:
said tip is substantially aligned with said upper cutting surface.

8. A scissors instrument according to claim 1, wherein:
said second blade includes a lower surface, and said tip is substantially parallel to said lower surface.

9. A scissors instrument according to claim 8, wherein:
said lower surface of said second blade tapers toward said tip.

10. A scissors instrument according to claim 8, wherein:
said lower surface of said second blade is substantially straight.

11. A scissors instrument according to claim 1, wherein:
said handle is a Castroviejos type handle.

12. A scissors instrument according to claim 1, wherein:
said handle includes at least one ring.

13. A scissors instrument according to claim 1, further comprising:
an elongate shaft proximal and distal ends, wherein said handle is coupled to said proximal end and said first and second blades are coupled adjacent said distal end.

14. A scissors instrument according to claim 13, wherein:
said shaft is tubular.

15. A scissor instrument according to claim 14, wherein:
said shaft is flexible.

16. A scissors instrument according to claim 15, wherein:
said shaft is a catheter.

17. A scissors instrument according to claim 1, wherein:
said tip has a cross-sectional shape which is one of,
i) round,
ii) triangular, and
iii) tear-drop.

18. A scissors instrument according to claim 1, wherein:
each of said first and second blades are tapered, and said second blade includes a secondary taper which at least partially defines said tissue piercing tip.

19. A scissors instrument according to claim 1, wherein:
each of said first and second blades are tapered, and said second blade includes a step which at least partially defines said tissue piercing tip.

20. A scissors instrument, comprising:
a) scissors blades;
b) a handle operable to move at least one scissors blade relative to the other; and
c) a tissue-piercing tip having a substantially constant diameter in relation to said scissors blades.

21. A scissors instrument according to claim 20, wherein:
said tip extends beyond said scissors blades.

22. A scissors instrument according to claim 12, wherein:
said scissors blades includes first and second scissors blades, and said tip extends from said second scissors blade.

23. A scissors instrument according to claim 22, wherein:
said first blade, and second blade and tip extending therefrom are of substantially equal length.

24. A method of creating an incision in an anatomical vessel, comprising:
a) providing a single instrument including scissors blades and a tissue-piercing needle element;
b) inserting the tissue-piercing element through an anterior surface of the vessel prior to incising the anterior surface of the vessel with another instrument; and
c) cutting the vessel between the scissors blades.

25. A method according to claim 24, further comprising:
d) prior to said cutting, advancing a scissors blade in alignment with the tissue-piercing element into the vessel.

26. A method according to claim 24, wherein
said advancing occurs parallel to a length of the vessel.
